(19) United States
(12) Patent Application Publication
(10) Pub. No.: US 2013/0023649 A1
(43) Pub. Date: Jan. 24, 2013

(54) Method for Recovering Kunitz-Trypsin Inhibitor Proteins from a Soy Processing Stream

(71) Applicants: Karsten Keller, Hockessin, DE (US); Darrell Marsh, High Ridge, MO (US); Charles Schasteen, University City, MO (US); Barry Tulk, Creve Coeur, MO (US); Marlene Mekel, High Ridge, MO (US); Jennifer Wu, St. Charles, MO (US)

(72) Inventors: Karsten Keller, Hockessin, DE (US); Darrell Marsh, High Ridge, MO (US); Charles Schasteen, University City, MO (US); Barry Tulk, Creve Coeur, MO (US); Marlene Mekel, High Ridge, MO (US); Jennifer Wu, St. Charles, MO (US)

(21) Appl. No.: 13/519,842

(22) PCT Filed: Dec. 30, 2010
(86) PCT No.: PCT/US10/62594

§ 371 Date: Sep. 26, 2012

Related U.S. Application Data
(60) Provisional application No. 61/291,312, filed on Dec. 30, 2009.

(51) Int. Cl. C07K 1/14; C07K 1/26; C07K 1/30; C07K 1/36; C07K 1/16; C07K 1/34

(52) U.S. Cl. 530/378; 204/456

(57) ABSTRACT

A process for recovering and isolating a KTI product having a total KTI protein concentration of at least 95 wt. % from a processing stream is disclosed.

### CROSS REFERENCE TO RELATED APPLICATIONS

This application claims priority from U.S. Provisional Application Ser. No. 61/291,312 filed on Dec. 30 2009, which is hereby incorporated by reference in its entirety.

### FIELD OF THE INVENTION

The present disclosure provides processes for the recovery of purified Kunitz-trypsin inhibitor (KTI) proteins from a soy processing stream. Specifically, the present disclosure provides processes comprising chromatographic separation and, optionally, one or more separation techniques for isolating and removing a KTI product that has a purity as represented by a total KTI protein concentration of at least about 95 wt. %.

### BACKGROUND OF THE INVENTION

Proteins that inhibit proteolytic enzymes are often found in high concentrations in many seeds and other plant storage organs. Inhibitor proteins are also found in virtually all animal tissues and fluids. These proteins have been the object of considerable research for many years because of their ability to complex with and inhibit proteolytic enzymes from animals and microorganisms. The inhibitors have become valuable tools for the study of proteolysis in medicine and biology. Protease inhibitors are of particular interest due to their therapeutic potentials in controlling proteinases involved in a number of disorders such as pancreatitis, shock, and emphysema, and as agents for the regulation of mammalian fertilization.

Soy processing streams contain a significant amount of protease inhibitors. Protease inhibitors are known to at least inhibit trypsin, chymotrypsin and potentially a variety of other key transmembrane proteases that regulate a range of key metabolic functions. Topical administration of protease inhibitors finds use in such conditions as atopic dermatitis, a common form of inflammation of the skin, which may be localized to a few patches or involve large portions of the body. The depigmenting activity of protease inhibitors and their capability to prevent ultraviolet-induced pigmentation have been demonstrated both in vitro and in vivo (See e.g., Paine et al., J. Invest. Dermatol., 116: 587-595 [2001]). Protease inhibitors have also been reported to facilitate wound healing. For example, secretory leukocyte protease inhibitor was demonstrated to reverse the tissue destruction and speed the wound healing process when topically applied. In addition, serine protease inhibitors can also help to reduce pain in lupus erythematosus patients (See e.g., U.S. Pat. No. 6,537,968).

Naturally occurring protease inhibitors can be found in a variety of foods such as cereal grains (oats, barley, and maize), brussels sprouts, onion, beetroot, wheat, finger millet, and peanuts. One source of interest is the soybean.

Two broad classes of protease inhibitor superfamilies have been identified from soybean and other legumes with each class having several isoinhibitors. Kunitz-trypsin inhibitor (KTI) is major member of the first class whose members have approximately 170-200 amino acids, molecular weights between 20-25 kDa, and act principally against trypsin. Kunitz-trypsin proteinase inhibitors are mostly single chain polypeptides with 4 cysteines linked in two disulfide bridges, and with one reactive site located in a loop defined by disulfide bridge. The second class of inhibitors contains 60-85 amino acids, has a range in molecular weight of 6-10 kDa, has a higher number of disulfide bonds, is relatively heat-stable, and inhibits both trypsin and chymotrypsin at independent binding sites. Bowman-Birk inhibitor (BBI) is an example of this class. The average level of protease inhibitors present in soybeans is around 1.4% and 0.6% for KTI and BBI, respectively, two of the most important protease inhibitors. Notably, these low levels make it impractical to isolate the natural protease inhibitor for clinical applications.

The prior art has not described a KTI product having a high purity level that is obtained from a soy protein source, without acid or alcohol extraction, or acetone precipitation. Methods disclosed in the art for isolating KTI products have been described in the following specific references but have not resulted in the isolation of a highly pure KTI product: J. Agric. Food Chem. 39(5):862-866 (1991); Protein Expression and Purification 30:167-170 (2003); J. Agric. Food Chem, 57(15): 7022-7029(2009); FEBS Letters 294(1,2):141-143 (1991); Journal of Food Science, 54(3):606-617 (1989); Journal of Agriculture and Food Chemistry, 49(3):1069-1086 (2001); Journal of Agriculture and Food Chemistry, 35(2):205-209 (1987); Yamamoto, M. and Ikenaka, T., Studies on Soybean Trypsin Inhibitors. I. Purification and Characterization of Two Soybean Trypsin Inhibitors, J. Biochem. (Tokyo), 62(2), 141-149 (1967); Birk, Y., The Bowman-Birk inhibitor: trypsin- and chymotrypsin-inhibitor from soybeans, Int. J. Peptide Protein Res., 25(2). 113-131 (1985); Hwang, D., Davis Lin, K. T., Yang, W., Foard, D., Purification, Partial Characterization, and Immunological Relationships of Multiple Low Molecular Weight Protease Inhibitors of Soybean, Biochimica et Biophysica Acta, 495, 369-382 (1977); Frattali, V., Soybean Inhibitors-Ill. Properties of a low molecular weight soybean proteinase inhibitor, Journal of Biological Chemistry, 244(2), 274-280 (1969); Kassell, B., Trypsin and Chymotrypsin Inhibitors from Soybean, Methods in Enzymology, 66(c), 853 (1970); Birk, Y., Proteinase Inhibitors from Legume Seeds, Methods in Enzymology, 57, 697; and Birk, Y., Trypsin and Chymotrypsin Inhibitors from Soybeans, Methods in Enzymology, 58, 700. Thus, there is a need for methods and compositions suitable for the production of high purity protease inhibitors and variants, specifically KTI.

Accordingly, the present invention describes processes for isolating a KTI product in high purity form through methods disclosed herein.

### SUMMARY OF THE INVENTION

The present invention describes novel methods that produce compositions that are highly enriched in KTI proteins. More particularly, the present invention describes the concentration of a soy processing stream through a series of sequential separation operations to remove various other components from the soy whey to yield a purified fraction suitable as a substrate for recovery of KTI proteins. As further detailed elsewhere herein, the present invention describes the recovery of a purified fraction from the soy processing stream through a process comprising one or more membrane separation (filtration) operations or chromatographic separation operations designed for recovery of the various components of the aqueous soy whey. Removal of the various components of the soy processing stream typically comprises concentration of the soy processing stream prior to and/or during removal of the components thereof.

Typically a purified fraction is prepared by removal of one or more impurities (e.g. microorganisms or minerals), one or more soy storage proteins (i.e. glycinin and β-conglycinin), one or more soy whey proteins, and/or one or more sugars from the soy processing stream. Recovery of KTI proteins in high purity is improved by removal of other major components of the processing stream (e.g. storage proteins, minerals, lipids, microorganisms, and sugars) that detract from purity by diluents, while likewise improving total KTI protein concentration by purifying the protein fraction through removal of components that are antagonists to the proteins and/or have deleterious effects (e.g. endotoxins). Specifically, the present disclosure provides processes that result in the isolation and removal of a KTI product that has purity represented by a total KTI protein concentration of at least about 95 wt. %.

The processes of the present invention comprise first introducing a soy processing stream into a filtration feed zone in contact with one side of a separation membrane; passing the fluid through the membrane to yield a first retentate comprising at least one soy storage protein and at least one soy whey protein in the filtration feed zone, and a first permeate comprising one or more sugars, water, and one or more minerals in the permeate zone; introducing the first retentate or a fraction thereof into a second filtration feed zone in contact with one side of a second separation membrane; passing the fluid through the second membrane to yield a second retentate comprising one or more soy storage proteins in the second filtration feed zone and a second permeate comprising water, one or more soy whey proteins, and sugars in the second permeate zone; introducing the protein concentrated soy processing stream into an ion exchange unit comprising an ion exchange resin contained therein; dividing the soy whey stream to provide a plurality of feed fractions for ion exchange; introducing the plurality of feed fractions into an ion exchange zone; contacting each of the plurality of feed fractions with an ion exchange resin to remove the KTI product by affinity for the ion exchange resin; and, contacting the ion exchange resin with an eluting medium to remove the KTI product from the ion exchange resin to form an eluted KTI protein fraction.

The present disclosure further encompasses a method using a variety of techniques that first separate water from the soy whey solids, followed by techniques that separate microorganisms from the soy whey solids, followed by techniques that separate proteins from the soy whey solids, followed by techniques that separate sugars from the soy whey solids, followed finally by the formation of a purified stream comprising water. While the order of steps disclosed herein provides one particular embodiment of the present invention, it should be noted that the steps disclosed herein may be performed in any order.

### BRIEF DESCRIPTION OF THE DRAWINGS

FIG. 1A is a schematic flow sheet depicting Steps 0 through 4 in a process for recovery of a purified soy whey protein from processing stream.

FIG. 1B is a schematic flow sheet depicting Steps 5, 6, 14, 15, 16, and 17 in a process for recovery of a purified soy whey protein from processing stream.

FIG. 1C is a schematic flow sheet depicting Steps 7 through 13 in a process for recovery of a purified soy whey protein from processing stream.

FIG. 1D is a schematic flow sheet depicting a process for recovery of a purified KTI product from an aqueous soy whey stream.

FIG. 2 depicts a sodium dodecyl sulfate polyacrylamide gel electrophoresis (SDS-PAGE) analysis of the Poros HS50 ion exchange fractions of KTI.

FIG. 3 depicts a SDS-PAGE analysis of the enriched KTI fraction.

FIG. 4 depicts a SDS-PAGE analysis of HiTrap DEAE FF ion exchange fractions.

FIG. 5 depicts a KTI band submitted for LC-MS/MS analysis.

FIG. 6 depicts a SDS-PAGE analysis of Mimo6HE fractions.

### DETAILED DESCRIPTION OF THE PREFERRED ASPECTS

Described herein are novel processes for recovering highly purified KTI proteins and other products from a variety of leguminous plant processing streams (including soy whey streams and soy molasses streams) generated in the manufacture of soy protein isolates. For example, the processes of the present disclosure comprise one or more separation techniques or methods (e.g. chromatographic separation or membrane separation) selected and designed to provide recovery of the KTI proteins or other products, or separation of various components of the soy whey stream, or both. Recovery of KTI proteins and one or more other components of the soy whey stream (e.g. various sugars, including oligosaccharides) may utilize a plurality of separation techniques, (e.g. membrane, chromatographic, precipitation, centrifugation, or filtration). The specific separation technique will depend upon the desired component to be recovered by separating it from other components of the processing stream.

For example, a purified KTI fraction is typically first prepared by removal of one or more impurities (e.g. microorganisms or minerals), followed by removal of additional impurities including one or more soy storage proteins (i.e. glycinin and β-conglycinin), followed by removal of one or more soy whey proteins, (including, for example, BBI and other non-KTI proteins or peptides), and/or followed by removal of one or more additional impurities including sugars from the soy whey. Recovery of KTI proteins in high purity form is improved by removal of other major components of the whey stream (e.g. storage proteins, minerals, and sugars) that detract from purity by diluents, while likewise improving purity by purifying the protein fraction through removal of components that are antagonists to the proteins and/or have deleterious effects (e.g. endotoxins). Removal of the various components of the soy whey typically comprises concentration of the soy whey prior to and/or during removal of the components of the soy whey. The methods of the present invention also will reduce pollution generated from processing large quantities of aqueous waste.

Removal of storage proteins, sugars, minerals, and other impurities yields fractions that are enriched in the desired KTI proteins and free of impurities that may be antagonists or toxins, or may otherwise have a deleterious effect. For example, typically a soy storage protein-enriched fraction may be recovered, along with a fraction enriched in one or more soy whey proteins. A fraction enriched in one more sugars (e.g. oligosaccharides and/or polysaccharides) is also typically prepared. Thus, the present methods provide a fraction that is suitable as a substrate for recovery of KTI proteins, and also provide other fractions that can be used for recovery of other useful products from aqueous soy whey. For example, removal of sugars and/or minerals from the soy whey stream produces a useful fraction from which the sugars can be further separated, thus yielding additional useful fractions: a concentrated sugar and a mineral fraction (that may include citric acid), and a relatively pure aqueous fraction that may be disposed of with minimal, if any, treatment or recycled as process water. Process water thus produced may be especially useful in practicing the present methods. Thus, a further advantage of the present methods may be reduced process water requirements as compared to conventional isolate preparation processes.

Methods of the present disclosure provide advantages over conventional methods for manufacture of soy protein isolates and concentrates in at least two ways. As noted, conventional methods for manufacturing soy protein materials typically dispose of the soy whey stream (e.g. aqueous soy whey or soy molasses). Thus, the products recovered by the methods of the present disclosure represent an additional product, and a revenue source not currently realized in connection with conventional soy protein isolate and soy protein concentrate manufacture. Furthermore, treatment of the soy whey stream or soy molasses to recover saleable products preferably reduces the costs associated with treatment and disposal of the soy whey stream or soy molasses. For example, as detailed elsewhere herein, various methods of the present invention provide a relatively pure processing stream that may be readily utilized in various other processes or disposed of with minimal, if any, treatment, thereby reducing the environmental impact of the process. Certain costs exist in association with the methods of the present disclosure, but the benefits of the additional product(s) isolated and minimization of waste disposal are believed to compensate for any added costs.

### A. Add-soluble Proteins

Soy protein isolates are typically precipitated from an aqueous extract of defatted soy flakes or soy flour at the isoelectric point of soy storage proteins (e.g. a pH of about 4.5). Thus, soy protein isolates generally include proteins that are not soluble in acidic liquid media. Similarly, the proteins of soy protein concentrates, the second-most refined soy protein material, are likewise generally not soluble in acidic liquid media. However, soy whey proteins recovered by the processes of the present disclosure are generally acid-soluble, meaning they are soluble in acidic liquid media.

For example, the present disclosure provides soy protein compositions derived from an aqueous soy whey and exhibiting advantageous solubility across a relatively wide range of pH of the aqueous (typically acidic) medium (e.g. an aqueous medium having a pH of from about 2 to about 10, from about 2 to about 7, or from about 2 to about 6) at ambient conditions (e.g. a temperature of about 25° C.). Typically the solubility of the soy protein composition is at least about 10 grams per liter (g/L), more typically at least about 15 g/L and, still more typically, at least about 20 g/L, It is to be understood that reference to solubility across a pH range (including in the appended claims) indicates that the specified solubility is achieved at any and all pH values falling within the specified pH range. For example, reference to a solubility of at least about 10 g/L across of a pH range of from about 2 to about 10 indicates that the specified solubility is achieved at a pH of 3, 4, 5, 6, etc.

Recovery of acid-soluble soy proteins by the processes of the present disclosure represents a significant advance in the art. As noted herein, the acid-soluble proteins are recovered from the soy whey stream which is typically discarded.

### B. Kunitz-Trypsin Protease Inhibitors

As discussed herein, soy processing streams, which include for example, soy whey stream and soy molasses stream, contain Kunitz-trypsin Inhibitor (KTI) proteins. This protease inhibitor is known to at least inhibit trypsin, and potentially a variety of other key proteases that regulate a range of key metabolic functions.

The KTI proteins isolated in accordance with the present embodiment may comprise a polypeptide having an amino acid sequence at least 50%, 60%, 65%, 70%, 75%, 80%, 85%, 90%, 95%, 99% or even 100% identical to SEQ ID NO: 1. In one embodiment, the KTI protein may comprise an amino acid sequence at least 70% identical to SEQ ID NO: 1, more preferably at least 80% identical to SEQ ID NO: 1, even more preferably at least 90% identical to SEQ ID NO: 1, and most preferably at least 95% identical to SEQ ID NO: 1.

In another aspect of the present embodiment, the amino acid sequence is at least 50%, 60%, 65%, 70%, 75%, 80%, 85%, 90%, 95%, 99%, or even 100% identical to SEQ ID NO: 1.

In certain aspects of the invention, sequence identity between two amino acid sequences is determined by comparing the amino acid sequences. In other aspects of the invention, sequence identity can be determined by comparing the amino acid sequences and its conserved amino acid substitutes. In certain aspects of the invention, a protein of the invention can have one or more conservative substitutions. In other aspects of the invention, a protein of the invention can have one or more non-conservative substitutions.

Naturally occurring amino acids include, for example, alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), and valine (V).

Conservative and non-conservative amino acid substitutions are known to those of ordinary skill in the art, for example, substituting an acidic amino acid for another acid amino acid may be considered a conservative substitution whereas substituting a basic amino acid for an acidic amino acid may be considered a non-conservative substitution; similarly, substituting a polar amino acid for another polar amino acid may be considered a conservative substitution whereas substituting a nonpolar amino acid for a polar amino acid may be considered a non-conservative substitution. Amino acids are generally grouped into the following categories (which can be used as a guide for determining whether a substitution is conservative or non-conservative): (1) polar/hydrophilic: N, Q, 5, T, K, R, H, D, E, C, and Y; (2) non-polar/hydrophobic: G, A, L, V, I, P, F, W, and M; (3) acidic: D, E, and C; (4) basic: K, R, and H; (5) aromatic: F, W, Y, and H; and (6) aliphatic: G, A, L, V, I, and P.

In certain aspects of the invention wherein one or more amino acid sequences are not identical to SEQ ID NO: 1, such one or more amino acid sequences also function as a KTI protein, which are known to inhibit trypsin activity. Methods for ascertaining these functions are described herein and are known to one of ordinary skill in the art.

In other aspects of the invention wherein a composition comprises one or more amino acid sequences that are not identical to SEQ ID NO: 1, such one or more amino acid sequences also function as a KTI protein, which are known to inhibit trypsin. Methods for ascertaining these functions are described herein and are known to one of ordinary skill in the art.

KTI proteins are comprised of 170 to 200 amino acid residues and approximately two disulfide bridges. The primary structure of a KTI protein has been known since 1973 (see Koide, T., and Ikenaka, T., Studies on Soybean Trypsin Inhibitors. 3. Amino-Acid Sequence of the Carboxyl-Terminal Region and the Complete Amino-Acid Sequence of Soybean Trypsin Inhibitor, Eur. J. Biochem. 32, 417, 1973).

The purity of the KTI products of the present disclosure represents previously unachieved levels of purity as compared to other KTI products. The purity of the KTI fraction is a function of total KTI protein concentration, specific activity (as measured by trypsin inhibitor units/g protein), and the absence of components that function as antagonists for KTI, toxins, or other components that have deleterious effect beyond merely diluting the efficiency per unit quantity of the KTI. Generally, the total KTI protein concentration of KTI products of the present disclosure is at least about 70 wt. %, or at least about 80 wt. %, Typically, the total KTI protein concentration of the KTI products of the present disclosure is at least about 90 wt. %, preferably at least about 95 wt. %, and, more preferably, at least about 99 wt. %.

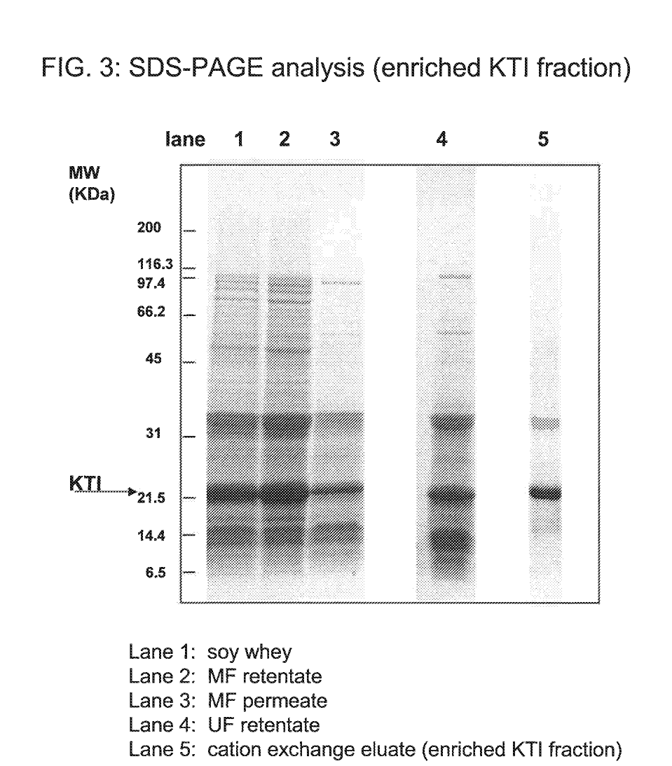

A “pure” monomeric protein will yield a single band after electrophoresis on a one- or two-dimensional SDS-PAGE gel, will elute from a gel filtration, high performance liquid chromatography (HPLC), or ion exchange column as a single symmetrical absorbance peak, will yield a single set of mass spectrometric, nuclear magnetic resonance (NMR), or W absorbance spectral signals, and where appropriate, will be free of contaminating enzyme activities. Since absolute purity can never be established, a simple criterion of purity is used routinely, namely, the inability to detect more than a single band of protein after SDS-PAGE. (See Mohan, Determination of purity and yield. Methods in Molecular Biology, 11, 307-323 (1992)). FIG. 3 depicts the KTI proteins of the present invention following one-dimensional gel electrophoresis. As FIG. 3 illustrates, the KTI proteins of the present invention showed as a single band corresponding to the molecular weight standard of 21 kDa and with different isoelectric points.

The purity of the KTI fraction is a function of total KTI protein concentration, specific activity (as measured by trypsin inhibitor units/g protein), and the absence of components that function as antagonists for KTI, toxins, or other components that have deleterious effect beyond merely diluting the efficiency per unit quantity of the KTI. Generally, the total KTI protein concentration of KTI products of the present disclosure is at least about 70 wt. %, or at least about 80 wt. %. Typically, the total KTI protein concentration of the KTI products of the present disclosure is at least about 90 wt. %, preferably at least about 95 wt. %, and, more preferably, at least about 99 wt. %.

Along with KTI purity, the total protein content of the KTI products of the present disclosure are advantageous and/or represent an advance over the art. KTI protein content of products of the present disclosure may be determined by conventional methods known in the art including, for example, the Lowry method described in Ohnishi, S. T., and Barr, J. K., A simplified method of quantitating proteins using the biuret and phenol reagents. Anal. Biochem., 86, 193 (1978) Generally, the total protein content of the KTI products of the present disclosure is at least about 60 wt. % (on a dry weight basis), at least about 70 wt. %, at least about 80 wt. %, or at least about 85 wt. %. Typically, the total protein content of KTI products of the present disclosure is at least about 90 wt. %, preferably at least about 95 wt. % and, more preferably, at least about 99 wt. %.

KTI proteins are known to only inhibit trypsin, while other components of the protein-containing composition (e.g. BBI proteins) are known to inhibit both trypsin and chymotrypsin. Thus, the absence of chymotrypsin inhibitor activity in the trypsin inhibiting preparations of the present invention indicates the presence of KTI proteins.

Similarly, trypsin inhibitor activity of KTI products of the present disclosure (expressed in terms of trypsin inhibitor units/g protein, or TIU/g protein) may be determined by conventional methods known in the art including, for example, in which one TIU is defined as the amount of a substrate which can inhibit 1 mg of trypsin and one trypsin unit equals ΔA410 of 0.019 per 10 minute with benzoyl-DL-arginine-p-nitroaniline (BAPA nitroaniline) as substrate at pH 8.2 and 37° C. Generally, the trypsin inhibitor activity of KTI products of the present disclosure is at least about 300 TIU/g protein, typically at least about 500 TIU/g protein, more typically at least about 950 TIU/g protein, and even more typically, at least about 1500 TIU/g protein.

Various applications for which the KTI products are currently believed to be suitable require relatively low endotoxin content. For example, various therapeutic applications require that the KTI product satisfy the applicable regulations for pharmaceutical-grade materials. Thus, in various preferred aspects, the total endotoxin content of the KTI product is preferably no more than about 1.5 EU/gm protein, more preferably no more than about 1 EU/gm protein, still more preferably no more than about 0.5 EU/gm protein and, even more preferably, no more than about 0.25 EU/gm protein. For example, in accordance with various such aspects, the total endotoxin content of the KTI product is typically from about 0.1 to about 1.5 EU/gm protein, more typically from about 0.1 to about 1.0 EU/gm protein and, still more typically, from about 0.1 to about 0.5 EU/gm protein (e.g. from about 0.1 to about 0.25 EU/gm protein). Additionally or alternatively, the total endotoxin content of the KTI products of the present disclosure is no more than about 5.0 endotoxin units per gram protein (EU/gm protein), or no more than about 2.5 EU/gm protein. For example, in various aspects, the total endotoxin content is from about 0.1 to about 5.0 EU/gm protein, or from about 0.1 to about 2.5 EU/gm protein.

It is to be understood that KTI products of the present disclosure may exhibit one, a combination, or all of the above-specified features. For example, KTI products of the present disclosure may exhibit the specified KTI purity and total protein contents. KTI products may also exhibit the specified KTI purity, trypsin inhibitor activity, and sequences disclosed herein. By way of further example, the KTI products may exhibit the specified KTI protein concentration and total endotoxin content. In these and still further aspects, the KTI products of the present disclosure may exhibit the specified total soy protein concentration and trypsin inhibitor activity. By way of further example, KTI products of the present disclosure may exhibit the specified total soy protein concentration and total endotoxin content. These combinations of properties of the KTI products are exemplary and this list is not intended to be exhaustive. That is, in accordance with the present disclosure, KTI products may exhibit any combination of the above-noted properties, at any of the above-specified values of within any of the above-specified ranges.

KTI products of the present invention can be obtained from any source or any process which allows for the separation, isolation, or purification of KTI from a native plant-based matrix. By way of non-limiting example, a native plant-based matrix can be derived from leguminous plants, including for example, soybeans, corn, peas, canola, sunflowers, sorghum, rice, amaranth, potato, tapioca, arrowroot, canna, lupin, rape, wheat, oats, rye, barley, peanut, jack bean, Job's tears, pea family legumes, Baru, lablab beans, lancepods apple leaf seed), alfalfa, snail medic seeds, lima beans, butter beans, kidney beans, bush beans, sugar cane, millet, timber tree, spinach, chapule, ciliates, dessert banana, lentil, bran, broad or fava bean, mung bean, adzuki bean, cow pea, jatropha, green algae, and combinations thereof. In particular aspects of the invention, KTI is obtained from soy in various processing streams. Various soy processing streams include, for example, an aqueous soy extract stream (which is any stream in which the protein components of a soy stream are in the soluble form, such as from a defatted soy material), an aqueous soymilk extract stream (which is any stream from a whole or partially defatted soy material in which the protein components of a soy stream are in the soluble form), an aqueous soy whey stream (which is any whey stream resulting from the precipitation or salting out of storage proteins; the precipitation method can include heat as well as chemical processes), an aqueous soy molasses stream (which is any stream generated by the removal of water from an aqueous soy whey stream), an aqueous soy protein concentrate soy molasses stream (which is any stream from the alcohol extraction of soluble sugars from the soy protein concentrate process), an aqueous soy permeate stream (which is any stream resulting from the separation of different molecular weight protein fractions where the smaller molecular weight proteins pass through a membrane), and an aqueous tofu whey stream (which includes any whey stream resulting from a tofu coagulation process). The amount of KTI product isolated by the processes of the present invention may be as small as a gram (lab scale isolation) or may be several metric tons (industrial or large scale isolation).

### C. Aqueous Whe Streams

Aqueous whey streams and molasses streams, which are types of soy processing streams, are generated from the process of refining a whole legume or oilseed. The whole legume or oilseed may be derived from a variety of suitable plants. By way of non-limiting example, suitable plants include leguminous plants, including for example, soybeans, corn, peas, canola, sunflowers, sorghum, rice, amaranth, potato, tapioca, arrowroot, canna, lupin, rape, wheat, oats, rye, barley, peanut, jack bean, Job's tears, pea family legumes, Baru, lablab beans, lancepods (e.g., apple leaf seed), alfalfa, snail medic seeds, lima beans, butter beans, kidney beans, bush beans, sugar cane, millet, timber tree, spinach, chapule, ciliates, dessert banana, lentil, bran, broad or fava bean, mung bean, adzuki bean, cow pea, jatropha, green algae, and combinations thereof. In one embodiment, the leguminous plant is soybean and the aqueous whey stream generated from the process of refining the soybean is an aqueous soy whey stream.

Aqueous soy whey streams generated in the manufacture of soy protein isolates are generally relatively dilute and are typically discarded as waste. More particularly, the aqueous soy whey stream typically has a total solids content of less than about 10 wt. %, typically less than about 7.5 wt. % and, still more typically, less than about 5 wt. %, For example, in various aspects, the solids content of the aqueous soy whey stream is from about 0.5 to about 10 wt. %, from about 1 wt. % to about 4 wt. %, or from about 1 to about 3 wt. % (e.g. about 2 wt. %). Thus, during commercial soy protein isolate production, a significant volume of waste water that must be treated or disposed is generated.

Soy whey streams typically contain a significant portion of the initial soy protein content of the starting material soybeans. As used herein the term “soy protein” generally refers to any and all of the proteins native to soybeans. Naturally occurring soy proteins are generally globular proteins having a hydrophobic core surrounded by a hydrophilic shell. Numerous soy proteins have been identified including, for example, storage proteins such as glycinin and β-conglycinin. Soy proteins likewise include protease inhibitors, such as the above-noted KTI proteins. Soy proteins also include hemagglutinins such as lectin, lipoxygenases, p -amylase, and lunasin. It is to be noted that the soy plant may be transformed to produce other proteins not normally expressed by soy plants. It is to be understood that reference herein to “soy proteins” likewise contemplates proteins thus produced.

On a dry weight basis, soy proteins constitute at least about 10 wt. %, at least about 15 wt. %, or at least about 20 wt. % of the soy whey stream (dry weight basis). Typically, soy proteins constitute from about 10 to about 40 wt. %, or from about 20 wt. % to about 30 wt. % of the soy whey stream (dry weight basis). Soy protein isolates typically contain a significant portion of the storage proteins of the soybean. However, the soy whey stream remaining after isolate precipitation likewise contains one or more soy storage proteins.

In addition to the various soy proteins, the aqueous soy whey stream likewise comprises one or more carbohydrates (i.e. sugars). Generally, sugars constitute at least about 25%, at least about 35%, or at least about 45% by weight of the soy whey stream (dry weight basis). Typically, sugars constitute from about 25% to about 75%, more typically from about 35% to about 65% and, still more typically, from about 40% to about 60% by weight of the soy whey stream (dry weight basis).

The sugars of the soy whey stream generally include one or more monosaccharides, and/or one or more oligosaccharides or polysaccharides. For example, in various aspects, the soy whey stream comprises monosaccharides selected from the group consisting of glucose, fructose, and combinations thereof. Typically, monosaccharides constitute from about 0.5% to about 10 wt. % and, more typically from about 1% to about 5 wt. % of the soy whey stream (dry weight basis). Further in accordance with these and various other aspects, the soy whey stream comprises oligosaccharides selected from the group consisting of sucrose, raffinose, stachyose, and combinations thereof. Typically, oligosaccharides constitute from about 30% to about 60% and, more typically, from about 40% to about 50% by weight of the soy whey stream (dry weight basis).

The aqueous soy whey stream also typically comprises an ash fraction that includes a variety of components including, for example, various minerals, phytic acid, citric acid, and vitamins. Minerals typically present in the soy whey stream include sodium, potassium, calcium, phosphorus, magnesium, chloride, iron, manganese, zinc, copper, and combinations thereof. Vitamins present in the soy whey stream include, for example, thiamine and riboflavin. Regardless of its precise composition, the ash fraction typically constitutes from about 5% to about 30% and, more typically, from about 10% to about 25% by weight of the soy whey stream (dry weight basis).

The aqueous soy whey stream also typically comprises a fat fraction that generally constitutes from about 0.1% to about 5% by weight of the soy whey stream (dry weight basis). In certain aspects of the invention, the fat content is measured by acid hydrolysis and is about 3% by weight of the soy whey stream (dry weight basis).

In addition to the above components, the aqueous soy whey stream also typically comprises one or more microorganisms including, for example, various bacteria, molds, and yeasts. The proportions of these components typically vary from about 100 to about 1×10⁹ colony forming units (CFU) per milliliter. As detailed elsewhere herein, in various aspects, the aqueous soy whey stream is treated to remove these component(s) prior to protein recovery and/or isolation.

As noted, conventional production of soy protein isolates typically includes disposal of the aqueous soy whey stream remaining after isolation of the soy protein isolate. In accordance with the present disclosure, recovery of one or more proteins and various other components (e.g. sugars and minerals) results in a relatively pure aqueous whey stream. Conventional soy whey streams from which the protein and one or more components have not been removed generally require treatment prior to disposal and/or reuse. In accordance with various aspects of the present disclosure the aqueous whey stream may be disposed of or utilized as process water with minimal, if any, treatment. For example, the aqueous whey stream may be used in one or more filtration (e.g. diafiltration) operations of the present disclosure.

In addition to recovery of KTI proteins from aqueous soy whey streams generated in the manufacture of soy protein isolates, it is to be understood that the processes described herein are likewise suitable for recovery of one or more components of soy molasses streams generated in the manufacture of a soy protein concentrate, as soy molasses streams are an additional type of soy processing stream.

### D. Recover of KTI Proteins

The processes described herein are directed to the recovery and isolation of purified KTI proteins present in an aqueous whey stream generated from the process of refining a whole legume or oilseed. As discussed hereinabove, the whole legume or oilseed may be derived from a variety of suitable plants. By way of non-limiting example, suitable plants include leguminous plants, including for example, soybeans, corn, peas, canola, sunflowers, sorghum, rice, amaranth, potato, tapioca, arrowroot, canna, lupin, rape, wheat, oats, rye, barley, peanut, jack bean, Job's tears, pea family legumes, Baru, lablab beans, lancepods (e.g., apple leaf seed), alfalfa, snail medic seeds, lima beans, butter beans, kidney beans, bush beans, sugar cane, millet, timber tree, spinach, chapule, ciliates, dessert banana, lentil, bran, broad or fava bean, mung bean, adzuki bean, cow pea, jatropha, green algae, and combinations thereof. In one embodiment, the leguminous plant is soybean and the aqueous whey stream generated from the process of refilling the soybean is an aqueous soy whey stream.

In various aspects, the present disclosure provides processes for recovery and isolation of KTI proteins present in an aqueous soy whey stream generated during soy protein isolate production. It should be noted that the processes of the present invention are not limited to soy whey or soy molasses streams and may be used to recover proteins and various other components from a wide variety of leguminous plant processing streams. In various aspects, fractions comprising a high proportion of KTI proteins are recovered from the soy whey stream.

Soy whey streams treated by the processes of the present disclosure are generally relatively dilute. To facilitate recovery and/or isolation of KTI proteins, the whey stream is preferably concentrated during the initial stage(s) of the process. Concentrating the soy whey stream aids in recovery and separation of KTI proteins from the whey stream. For example, in a preferred embodiment of the present disclosure, water is removed from the aqueous soy whey prior to recovery of KTI proteins by contacting the aqueous soy whey or a fraction thereof with a separation membrane to form a retentate comprising the aqueous soy whey and a permeate comprising water. In other embodiments of the present disclosure, water may be removed from the soy whey through any method known in the art, for example by evaporating.

Along with recovery of KTI proteins, processes of the present disclosure typically separate proteins from sugars present in the soy whey stream. Optionally, the processes of the present disclosure may be configured and controlled to separate the sugars of the soy whey stream into one or more fractions (e.g. a monosaccharide-rich fraction and/or an oligosaccharide-rich fraction). This may be done in multiple steps to separate different sugars from the proteins. Recovery of sugars from the soy whey stream thus provides a further product stream. As noted, sugar removal typically produces a fraction from which the sugars can be separated to yield both a concentrated sugar fraction and a relatively pure aqueous fraction that may be disposed of with minimal, if any, treatment or recycled as process water.

Following treatment of the retentate to remove sugars, the retentate is further treated to remove additional components. As noted, various soy whey streams that may be treated by the present disclosure include one or more minerals (e.g. phosphorus and calcium). It has been observed that the presence of one or more minerals may pose a challenge to downstream processing by, for example, membrane fouling and difficulty in separating from components desired to be recovered (i.e. KTI proteins). In addition to recovery of these desired components generally, removal of minerals from the soy whey is also currently believed to contribute to the recovery of KTI products having greater purity. As detailed elsewhere herein, mineral removal from the soy whey may generally proceed in accordance with methods known in the art including, for example, precipitation and centrifugation. Since phytic acid is typically present in the aqueous soy whey streams treated by the present processes, minerals such as calcium and magnesium are typically recovered in the form of calcium and magnesium phytates. Other minerals removed may also include, for example, sodium, potassium, zinc, iron, manganese, and copper.

The present disclosure encompasses a variety of processes suitable for recovery of KTI proteins from aqueous soy whey streams generated in the production of soy protein isolates. Generally, the processes of the present disclosure comprise one or more operations designed and configured to separate out the particular components of the aqueous soy whey stream, thereby concentrating the whey stream and enabling the recovery of purified KTI proteins there from.

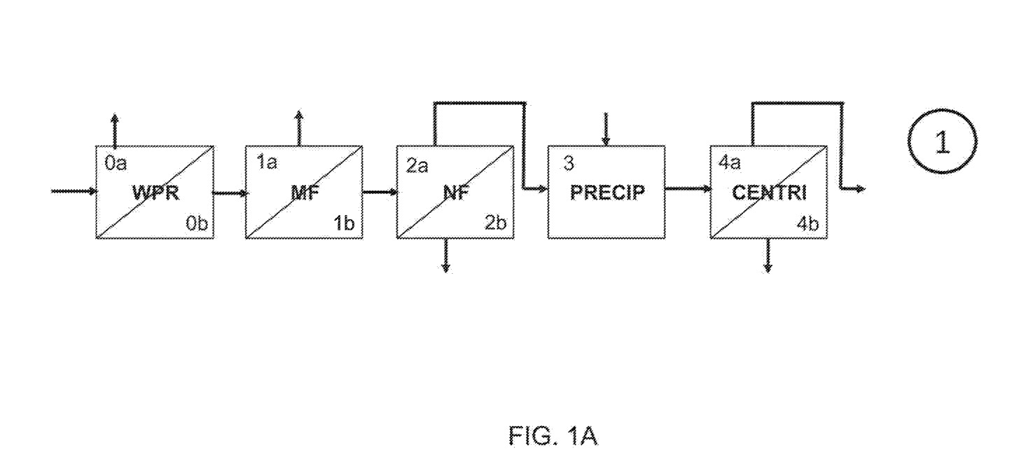

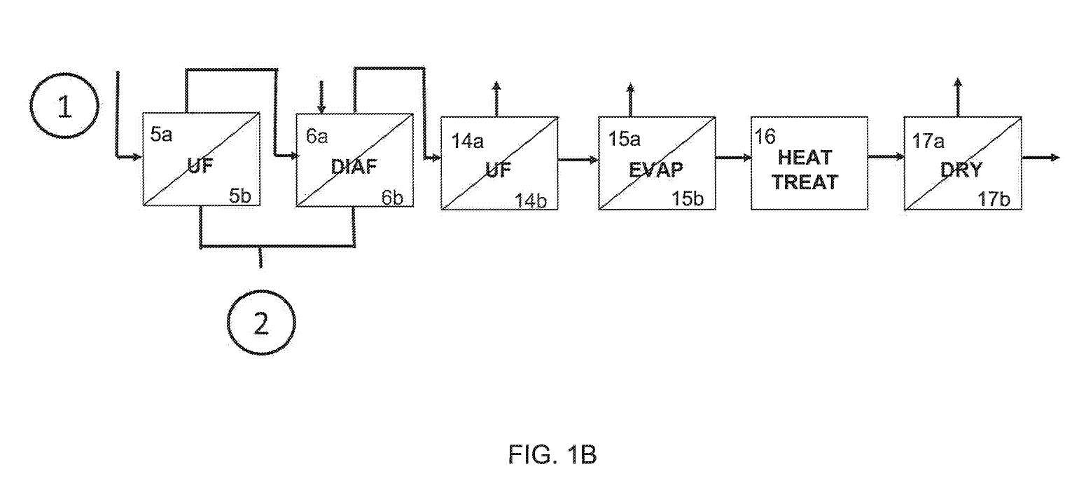

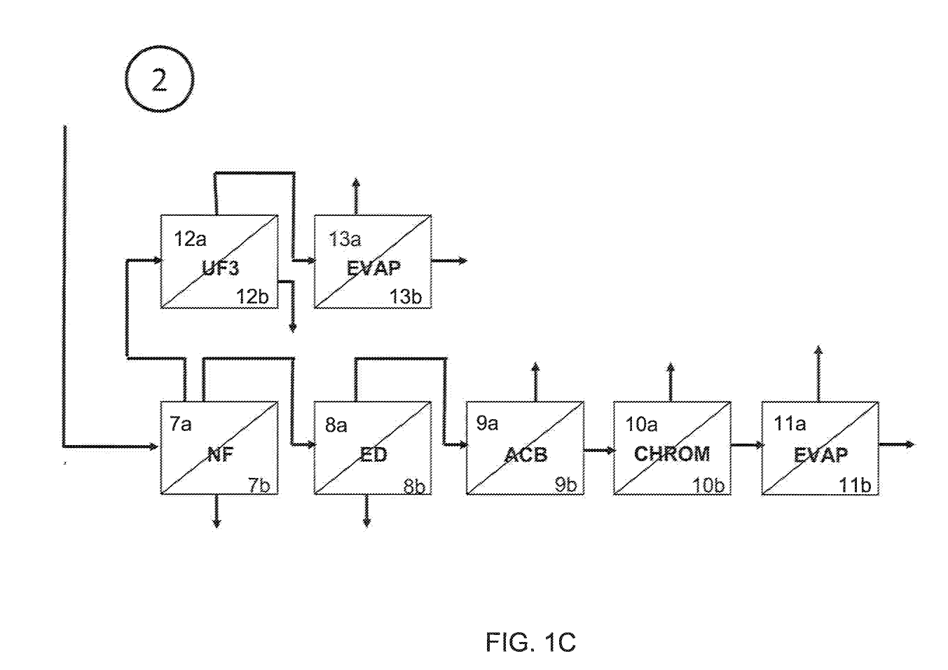

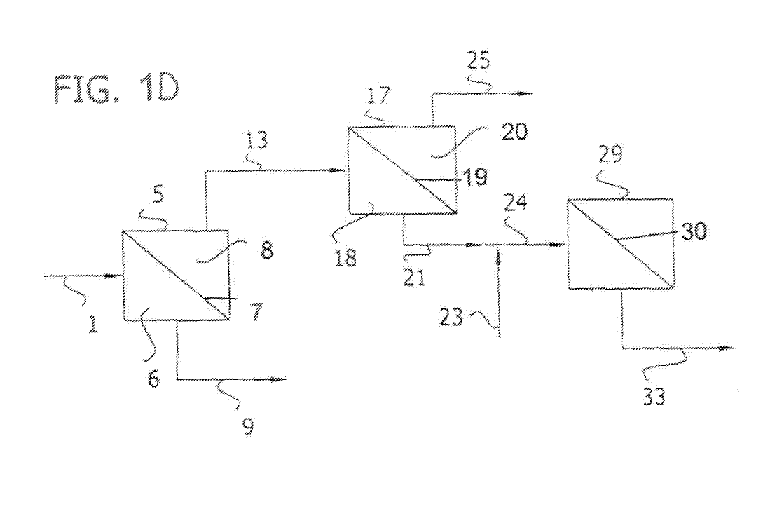

Generally, in accordance with the present disclosure, any of a variety of separation or purification techniques well-known in the art may be utilized to remove the various interfering components found in aqueous soy whey and isolate purified KTI proteins there from including, for example, membrane separation techniques (e.g. filtration, such as ultrafiltration, microfiltration, nanofiltration, and/or reverse osmosis), chromatographic separation techniques (e.g. ion exchange chromatography, membrane chromatography, adsorption chromatography, size exclusion chromatography, reverse phase chromatography, gel filtration, affinity chromatography, which include, for example, anion or cation exchange chromatography, simulated moving bed chromatography, expanded bed adsorption chromatography, gel filtration, reverse-phase chromatography, and/or mixed bed ion exchange), electrophoresis, dialysis, particulate filtration, precipitation, centrifugation, crystallization, and combinations thereof. A primary basis for separation of the various components is molecular size, although in filtration applications, the permeability of a filter medium can be affected by the chemical, molecular or electrostatic properties of the sample. As detailed elsewhere herein (e.g. below with reference to FIGS. 1A, 1B, 1C, and 1D), processes of the present disclosure typically utilize more than one type of separation membrane depending upon the particular component of the whey stream to be removed. For example, one step of the process may utilize an ultrafiltration separation membrane, followed by one or more steps utilizing a nanofiltration separation membrane.

In certain aspects for the removal of insoluble solids, particulate filtration, precipitation, centrifugation, crystallization, and combinations thereof may be used. Insoluble solids removed by these methods are typically greater than 20 μm.

Microfiltration is the process of separating solid particles from fluids by using a microfiltration membrane. Suitable microfiltration membranes are constructed of suitable materials known in the art including, for example, polysulfone, modified polysulfone, ceramic, and stainless steel. Microfiltration membranes typically have a pore size ranging from about 0.1 microns (μm) to about 20 μm. In certain aspects, microfiltration membranes have a pore size ranging from about 0.2 μm to about 2 μm.

Ultrafiltration is similar to microfiltration but differs in the pore size of the separation membrane. Ultrafiltration membranes are typically used to separate molecules having high molecular weights (including, for example, proteins) from molecules having lower molecular weights. Suitable ultrafiltration membranes are typically constructed of suitable materials known in the art, such as, for example polysulfone (PS), polyethersulfone (PES), polypropylene (PP), polyvinylidenefluoride (PVDF), regenerated cellulose (RC), ceramic, stainless steel, or thin-film composite. Ultrafiltration membranes typically have a molecular weight cut off (MWCO) of from about 1 to about 300 kilodaltons (kDa) or from about 5 to about 50 kDa. Additionally or alternatively, suitable ultrafiltration membranes may have a pore size of from about 0.002 μm to about 0.5 μm.

Nanofiltration is used to remove small molecules from fluids. Suitable nanofiltration membranes are typically constructed of suitable materials known in the art (e.g. polyethersulfone, polysulfone, ceramic and polyamide-type thin film composite on polyester) and typically have a MWCO of from about 0.1 to about 5 kDa or from about 1 to about 4 kDa. Additionally or alternatively, suitable nanofiltration membranes may have a pore size of from about 0.0009 μm to about 0.009 μm.

Reverse osmosis (or hyperfiltration) is typically used for the concentration of sugars. Suitable reverse osmosis membranes include those generally known in the art (e.g. membranes having a pore size of less than 0.5 nm).

The separation membranes utilized in the filtration steps of the present invention may be arranged in accordance with one or more configurations known in the art, alone or in combination. For example, the membranes may be configured in the form a flat plate, or cassette module in which layers of membrane are combined together (along with optional layers of separator screens). Aqueous soy whey is generally introduced into alternating channels at one end of the stack and fluid passes through the membrane into one or more filtrate, or permeate channels. The separation membranes may also be arranged in a spiral wound module in which alternating layers of membrane are wound around a hollow central core. Aqueous soy whey is introduced into one end of the module while fluid passes through the alternating layers of the membrane and toward and into the core of the module. By way of further example, the separation membrane may be arranged in a hollow fiber module comprising a bundle of relatively narrow membrane tubes. Aqueous soy whey is introduced into the module and fluid passes through the bundle of membrane tubes transverse the flow of soy whey through the module. Suitable membrane arrangements are described, for example, in U.S. Pat. No. 6,946,075, the entire contents of which are incorporated herein by reference.

The filtration steps of the present invention, as further described herein, may utilize direct (normal-flow) filtration or tangential (cross-flow) filtration. In direct or normal-flow filtration, fluid (i.e. aqueous soy whey) is conveyed directly toward a separation membrane. Alternatively, in tangential or cross-flow filtration, fluid (i.e. aqueous soy whey) may be conveyed tangentially along the surface of the separation membrane. One advantage of tangential, or cross-flow filtration is that the frictional or sweeping force exerted tangentially on the membrane by the flow of aqueous soy whey typically aids in maintaining flux rate. Accordingly, in various aspects, one or more steps, and combinations thereof, in the processes of the present disclosure are operated as cross-flow filtration. Suitable cross-flow filters include those generally known in the art, including those described in U.S. Pat. No. 6,946,075. It is to be understood that passage of fluid may suitably proceed in accordance with normal and/or tangential (i.e. cross) flow. It is to be further understood that passage of fluid through other membrane separation units detailed elsewhere herein in connection with the embodiments depicted in FIGS. 1A, 1B, 1C, and 1D, and other aspects, may proceed in accordance with either or both of these mechanisms.

The process described by the present invention involves selection of the appropriate separation operation or combination of operations to sequentially remove various constituents from the soy whey stream and recover or isolate a KTI product, which KTI product comprises a level of purity that has not been previously achieved in the art. Generally, and as detailed elsewhere herein, the processes for recovery of KTI proteins utilize a combination of membrane separation and chromatographic separation (e.g. ion exchange) operations.

As noted elsewhere herein, aqueous soy whey streams treated by the processes of the present disclosure are generally relatively dilute. In various aspects the aqueous soy whey is concentrated by, for example, removal of water, by a concentration factor of at least about 2 (e.g. about 3 or about 6) prior to recovery of targeted, individual proteins.

As compared to other methods for recovery of KTI proteins, recovery of KTI proteins by simulated moving bed (SMB) chromatography generally may also provide advantages of lower cost, throughput, and/or flexibility due, at least in part, to the adaptability for treatment of plurality of samples of aqueous soy whey.

It has been observed that one or more components of the soy whey stream may interfere with recovery of KTI proteins. For example, often during soy protein isolate manufacture, a silicon compound, typically a silicone, is introduced as a defoaming agent, usually in the form of a silicon-containing compound such as those commercially available from Hydrite Chemical or Emerald Performance Materials. Regardless of the precise source, organic silicon compounds are typically present in the soy whey stream at concentrations of up to about 15 parts per million (ppm), up to about 10 ppm, or up to about 5 ppm based on silicon content. The presence of organic silicon compounds is generally undesired as it may interfere with recovery of KTI proteins of the soy whey stream.

Accordingly, in various aspects, silicones and/or other organic silicon compounds are removed from the soy whey stream as detailed elsewhere herein prior to treatment for recover and separation of KTI proteins. Preferably, silicon compounds are removed as further detailed herein to such a degree that the soy whey contains no more than trace levels of organic silicon. Additionally or alternatively, the aqueous soy whey may comprise one or more microorganisms that may interfere with recovery of the desired components of the aqueous soy whey and/or are undesired in a final, recovered product of the process.

For removal of these interfering components the soy whey stream may be filtered using a separation membrane selective for retention of silicon defoaming agent and/or one or more microorganisms, to yield a retentate comprising silicon and/or one or more microorganisms and a permeate comprising the aqueous soy whey. The particular membrane (including, for example, microfiltration) used in this initial purification is selected in view of the component(s) to be removed. Regardless of the type of membrane selected and the component removed from the soy whey stream, preferably at least a substantial portion, and preferably substantially all, of the desired KTI protein is found in the retentate. Further in this regard, it is to be noted that reference to a permeate comprising the aqueous soy whey indicates that treatment of the whey stream for removal of one or more impurities has little, if any, impact on the other components of the soy whey stream.

In various alternative aspects, bacteria contained in the whey stream may be killed by heating prior to recovery of proteins. The manner of heating the soy whey stream for destroying bacteria is not narrowly critical and may generally be conducted in accordance with conventional methods known in the art.

It is understood by those skilled in the art of separation technology that there can be residual components in each stream since separation is never 100%. Further, one skilled in the art realizes that separation technology can vary depending on the starting raw material.

Step 0 (See FIG. 1A)—Whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between 3.5 and 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 1 (See FIG. 1A)—Microbiology reduction can start with the product of the whey protein pretreatment step, including but not limited to pre-treated soy whey. This step involves microfiltration of the pre-treated soy whey. Process variables and alternatives in this step include but are not limited to, centrifugation, dead-end filtration, heat sterilization, ultraviolet sterilization, microfiltration, crossflow membrane filtration, and combinations thereof. Crossflow membrane filtration includes but is not limited to: spiral-wound, plate and frame, hollow fiber, ceramic, dynamic or rotating disk, nanofiber, and combinations thereof. The pH of step 1 can be between about 2.0 and about 12.0, or between about 3.5 and about 5.5, or about 5.3. The temperature can be between about 5° C. and about 90° C., or between about 25° C. and 75° C. or about 50° C. Products from step 1 include but are not limited to storage proteins, microorganisms, silicon, and combinations thereof in stream 1a and purified pre-treated soy whey in stream 1b.

Step 2 (See FIG. 1A)—A water and mineral removal can start with the purified pre-treated soy whey from stream 1b or 4a, or pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and 75° C., or about 50° C. Products from this water removal step include but are not limited to purified pre-treated soy whey in stream 2a and water, some minerals, monovalent cations and combinations thereof in stream 2b.

Step 3 (See FIG. 1A)—the mineral precipitation step can start with purified pre-treated soy whey from stream 2a or pretreated soy whey from streams 0a or 1b. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 1A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 5 (See FIG. 1B)—the protein separation and concentration step can start with purified pre-treated whey from stream 4a or the whey from streams 0a, 1b, or 2a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Step 6 (See FIG. 1B)—the protein washing and purification step can start with soy whey protein, BBI, KTI, storage proteins, other proteins or purified pre-treated whey from stream 4a or 5a, or whey from streams 0a, 1b, or 2a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., between about 25° C. and 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Step 7 (See FIG. 1C)—a water removal step can start with peptides, soy oligosaccharides, water, minerals, and combinations thereof from stream 5b and/or stream 6b. It includes a nanofiltration step. Process variables and alternatives in this step include but are not limited to, reverse osmosis, evaporation, nanofiltration, water diafiltration, buffer diafiltration, and combinations thereof. The pH of step 7 can be between about 2.0 and about 12.0, or between about 6.0 and about 9.0, or about 7.0. The temperature can be between about 5° C. and about 90° C., between about 25° C. and 75° C., or about 50° C. Products from stream 7a include but are not limited to, peptides, soy oligosaccharides, water, minerals, and combinations thereof. Products from stream 7b include but are not limited to, water, minerals, and combinations thereof.

Step 8 (See FIG. 1C)—a mineral removal step can start with peptides, soy oligosaccharides, water, minerals, and combinations thereof from streams 5b, 6b, 7a, and/or 12b. It includes an electrodialysis membrane step. Process variables and alternatives in this step include but are not limited to, ion exchange columns, chromatography, and combinations thereof. Processing aids that can be used in this mineral removal step include but are not limited to, water, enzymes, and combinations thereof. Enzymes include but are not limited to protease, phytase, and combinations thereof. The pH of step 8 can be between about 2.0 and about 12.0, or between about 6.0 and about 9.0, or about 7.0. The temperature can be between about 5° C. and about 90° C., between about 25° C. and 50° C., or about 40° C. Products from stream 8a include but are not limited to, de-mineralized soy oligosaccharides with conductivity between about 10 milli Siemens/centimeter (mS/cm) and about 0.5 mS/cm, or about 2 mS/cm. Products from stream 8b include but are not limited to, minerals, water, and combinations thereof,

Step 9 (See FIG. 1C)—a color removal step can start with de-mineralized soy oligosaccharides from streams 8a, 5b, 6b, 12b, and/or 7a). It utilizes an active carbon bed. Process variables and alternatives in this step include but are not limited to, ion exchange. Processing aids that can be used in this color removal step include but are not limited to, active carbon, ion exchange resins, and combinations thereof. The temperature can be between about 5° C. and about 90° C., or about 40° C. Products from stream 9a include but are not limited to, color compounds. Stream 9b is a decolored solution. Products from stream 9b include but are not limited to, soy oligosaccharides, and combinations thereof.

Step 10 (See FIG. 1C)—a soy oligosaccharide fractionation step can start with soy oligosaccharides, and combinations thereof from streams 9b, 5b, 6b, 7a, and/or 8a. It includes a chromatography step. Process variables and alternatives in this step include but are not limited to, chromatography, nanofiltration, and combinations thereof. Processing aids that can be used in this soy oligosaccharide fractionation step include but are not limited to acid or base to adjust the pH as one skilled in the art would know, based on the resin used. Products from stream 10a include but are not limited to, soy oligosaccharides. Products from stream 10b include but are not limited to soy oligosaccharides.

Step 11 (See FIG. 1C)—a water removal step can start with soy oligosaccharides from streams 9b, 5b, 6b, 7a, 8a, and/or 10b. It includes an evaporation step. Process variables and alternatives in this step include but are not limited to, evaporation, reverse osmosis, nanofiltration, and combinations thereof. Processing aids that can be used in this water removal step include but are not limited to, defoamer, steam, vacuum, and combinations thereof. The temperature can be between about 5° C. and about 90° C., or about 60° C. Products from stream 11a include but are not limited to, water. Products from stream 11b include but are not limited to, soy oligosaccharides.

Step 12 (See FIG. 1C)—an additional protein separation from soy oligosaccharides step can start with peptides, soy oligosaccharides, water, minerals, and combinations thereof from stream 7a, 5b, and/or 6b. It includes an ultrafiltration step. Process variables and alternatives in this step include but are not limited to, crossflow membrane filtration, ultrafiltration with pore sizes between about 50 kDa and about 1 kDa, and combinations thereof. Crossflow membrane filtration includes but is not limited to: spiral-wound, plate and frame, hollow fiber, ceramic, dynamic or rotating disk, nanofiber, and combinations thereof. Processing aids that can be used in this protein separation from sugars step include but are not limited to, acids, bases, protease, phytase, and combinations thereof. The pH of step 12 can be between about 2.0 and about 12.0, about 7.0. The temperature can be between about 5° C. and about 90° C., between about 25° C. and 75° C., or about 50° C. Products from stream 12b include but are not limited to, soy oligosaccharides, water, minerals, and combinations thereof. Products from stream 12a include but are not limited to, peptides, other proteins, and combinations thereof.

Step 13 (See FIG. 1C)—a water removal step can start with, peptides, and other proteins from stream 12a. It includes an evaporation step. Process variables and alternatives in this step include but are not limited to, reverse osmosis, nanofiltration, spray drying and combinations thereof. Products from stream 13a include but are not limited to, water. Products from stream 13b include but are not limited to, peptides, other proteins, and combinations thereof.

Step 14 (See FIG. 1B)—a protein fractionation step may be done by starting with soy whey protein, BBI, KTI, storage proteins, other proteins, and combinations thereof from streams 6a and/or 5a. It includes an ultrafiltration (with pore sizes from 300 kDa to 10 kDa) step. Process variables and alternatives in this step include but are not limited to, crossflow membrane filtration, ultrafiltration, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 14 can be between about 2.0 and about 12.0, or between about 6.0 and about 9.0, or about 7.0. The temperature can be between about 5° C. and about 90° C., between about 25° C. and 75° C., or about 50° C. Products from stream 14a include but are not limited to, storage proteins. Products from stream 14b include but are not limited to, soy whey protein, BBI, KTI, other proteins, and combinations thereof.

Step 15 (See FIG. 1B)—a water removal step can start with soy whey protein, BBI, KTI and, other proteins from streams 6a, 5a, and/or 14b. It includes an evaporation step. Process variables and alternatives in this step include but are not limited to, evaporation, nanofiltration, RO, and combinations thereof. Products from stream 15a include but are not limited to, water. Stream 15b products include but are not limited to soy whey protein, BBI, KTI, other proteins, and combinations thereof.

Step 16 (See FIG. 1B)—a heat treatment and flash cooling step can start with soy whey protein, BBI, KTI and, other proteins from streams 6a, 5a, 14b, and/or 15b. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature of the heating step can be between about 129° C. and about 160° C., or about 152° C. Temperature hold time can be between about 8 seconds and about 15 seconds, or about 9 seconds. Upon flash cooling, the temperature can be between about 50° C. and about 95° C., or about 82° C. Products from stream 16 include but are not limited to, soy whey protein.

Step 17 (See FIG. 1B)—a drying step can start with soy whey protein, BBI, KTI and, other proteins from streams 6a, 5a, 14b, 15b, and/or 16. It includes a drying step. The liquid feed temperature can be between about 50° C. and about 95° C., or about 82° C. The inlet temperature can be between about 175° C. and about 370° C., or about 290° C. The exhaust temperature can be between about 65° C. and about 98° C., or about 88° C. Products from stream 17a include but are not limited to, water. Products from stream 17b include but are not limited to, soy whey protein which includes, BBI, KTI and, other proteins.

FIG. 1D depicts an embodiment of a process of the present disclosure for recovery of one or more individual proteins from a soy whey stream generated in the production of soy protein isolate.

While not depicted in FIG. 1D, pretreatment of whey protein can start with processing streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of the pretreatment step can be between about 3.0 and about 6.0, and preferably 4.5. The temperature can be between about 70° C. and about 95° C., and preferably about 85° C. Temperature hold times can vary between about 0 minutes to about 20 minutes, and preferably about 10 minutes. Products from the pretreatment of the whey protein include but are not limited to soluble components in the aqueous phase of the whey stream (pretreated soy whey) (molecular weight of equal to or less than about 50 kiloDalton (kDa)) in the retentate and insoluble large molecular weight proteins (between about 300 kDa and between about 50 kDa) in the permeate, such as pre-treated soy whey, storage proteins, and combinations thereof.

As illustrated in FIG. 1D, the soy whey 1 (which may be pre-treated) is introduced into a membrane separation unit 5 comprising a first filtration feed zone 6 in contact with one side of a separation membrane 7 at a pressure higher than the pressure in a first permeate zone 8 on the other side of the membrane. Preferably, membrane separation unit 5 comprises at least one microfiltration membrane.

The transmembrane pressure across the separation membrane 7 within membrane separation unit 5 is generally at least about 1 psi, at least about 5 psi, at least about 10 psi, at least about 20 psi, at least about 30 psi, at least about 40 psi, at least about 50 psi, or at least about 75 psi. Fluid typically passes through the membrane at a volumetric flow, or flux of at least about 1 liter fluid/hour-m², or from about 1 to about 400 liters fluid/hour-m² cross-sectional membrane area transverse to the direction of flow. Flow rate may be affected by, for example, the type of filtration, fouling of membranes, etc. The soy whey is typically introduced into the filtration feed zone of the membrane separation unit at a temperature of from about 0° C. to about 100° C. and, more typically, at a temperature of from about 25° C. to about 75° C. Typically, aqueous soy whey 1 is reduced in volume by about 5% due to the retentate. Passage of fluid through the separation membrane results in a first retentate 9 and a first permeate 13 within first permeate zone 8. The first retentate 9 will primarily comprise one or more microorganisms and insoluble material, more particularly, the first retentate 9 typically is enriched in microorganisms relative to the first permeate 13. Preferably, the first retentate 9 contains a substantial portion, if not substantially all, of the microorganism content of the aqueous soy whey. Even more preferably, the first retentate 9 also comprises a substantial portion of the antifoam agent (e.g. silicon of the organic silicon- or lipid-containing compounds present in the aqueous soy whey) and, more particularly, preferably comprises at least about 70 wt. %, more preferably at least about 80 wt. % and, still more preferably, at least about 90 wt. % of the antifoam agent content of the aqueous soy whey based on antifoam agent content. The first permeate 13 will primarily comprise all of the various remaining components of the aqueous soy whey stream, such as the soluble soy storage proteins, soy whey proteins, various sugars, water, minerals, isoflavones, and vitamins.

Again with reference to FIG. 1D, the first permeate 13 is introduced into membrane separation unit 17 comprising a second filtration feed zone 18 in contact with one side of a separation membrane 19 at a pressure higher than the pressure in a second permeate zone 20. Membrane separation unit 17 preferably comprises at least one ultrafiltration membrane as the separation membrane 19. The transmembrane pressure across the separation membrane 19 within membrane separation unit 17 is generally at least about 5 psi, at least about 10 psi, at least about 25 psi, at least about 50 psi, at least about 100 psi, or at least about 150 psi. Fluid typically passes through the membrane at a volumetric flow, or flux, of at least about 1 liter fluid/hour-m², or from about 1 to about 150 liters fluid/hour-m² cross-sectional membrane area transverse to the direction of flow. The soy whey is typically introduced into the filtration feed zone of the membrane separation unit at a temperature of from about 0° C. to about 100° C. and, more typically, at a temperature of from about 25° C. to about 75° C. Typically, aqueous soy whey 1 is concentrated by a concentration factor of at least about 5, or from about 5 to about 75 (e.g. about 25). The ultrafiltration step may optionally include diafiltration. Diafiltration volumes may typically range from about 1 up to about 10 parts diafiltration volume per part of retentate.

Passage of fluid through the separation membrane results in a second retentate 21 and a second permeate 25. The second retentate 21 comprises a significant fraction of the protein content of the aqueous soy whey and, thus, is further treated for recovery of KTI proteins. Preferably, the second retentate 21 comprises at least about 25 wt. % to at least about 90 wt. % (e.g. at least about 50 wt. %) (dry weight basis) of various soy whey proteins present in the aqueous soy whey introduced into the first filtration feed zone 6.

Again with reference to FIG. 1D, the second permeate 25 generally comprises any proteins not recovered in second retentate 21 and various other components of the soy whey stream (e.g. various sugars, water, minerals, vitamins, and isoflavones). Although not illustrated in FIG. 1D, the components of the second permeate 25 may be further processed according to suitable separation operations in order to isolate and/or remove the individual components from the aqueous whey stream. Following the additional separation steps, a relatively pure water stream will preferably be formed, requiring minimal, if any, treatment prior to disposal or use. Therefore, the invention described herein also processes environmental benefits by, for example, improving environmental quality.

The second retentate 21 is combined with a carrier stream 23 to form the feed 24 to the ion exchange column or unit 29 containing at least one ion exchange resin 30. The precise composition of the carrier stream is not narrowly critical. Typically, the pH of the carrier stream is from about 1 to about 7, more typically from about 2 to about 6 and, still more typically, from about 2 to about 5 even more typically from about 3 to about 4. In various aspects for recovery of KTI proteins, the carrier stream comprises a non-volatile buffer, including, for example sodium citrate or a volatile buffer including, for example, ammonium formate. For example, in various aspects, the carrier stream comprises a counter ion containing buffer in an aqueous mixture at a concentration of from about 10 to about 30 millimolar (e.g. 20 mM).

The pH of the second retentate 21 and/or feed stream 24 affects solubility of soy proteins, and precipitated proteins may result in fouling of the ion exchange column. Thus, it may be desired to control the pH of the feed to the ion exchange column within certain limits (e.g. by buffering). If necessary, the pH of the feed may be maintained within the ranges by, for example, dilution of the second retentate, carrier stream, and/or the feed provided by the combination of the retentate and carrier stream. The composition of the diluent is not narrowly critical and is typically an aqueous medium (e.g. deionized water) that may be readily selected by one skilled in the art. In addition to impacting the pH of the feed, dilution also typically reduces the inherent ionic strength of the feed, which promotes binding of proteins to the ion exchange resin. Additionally or alternatively, the pH of the feed may be controlled by selection of the carrier stream.

The ion exchange resin is chosen to be suitable for selective retention and recovery of one or more proteins present in second retentate 21 and feed 24. In various aspects, the ion exchange resin is selected for selective retention of KTI proteins or retention of non-KTI proteins such that KTI proteins are separated from non-KTI proteins. The following discussion focuses on recovery and isolation of KTI proteins from an aqueous soy whey (i.e. second retentate 21). However, it is to be understood that the following procedure is readily adaptable to recovery of other target proteins (e.g. BBI proteins) as well as other types of incoming streams besides aqueous (e.g. reconstituted from spray dried).

Regardless of the precise configuration of the ion exchange unit, suitable ion exchange resins for recovery of KTI proteins include a variety of cation and anion exchange resins. Although both cation exchange resins and anion exchange resins are, depending on the feed to the ion exchange column, suitable for recovery of KTI proteins, in various aspects the ion exchange resin comprises a cation exchange resin. For example, a protein exposed to a pH below its isoelectric point (pI) is more likely to have regions of positive charge and, therefore, bind more tightly to a cation exchange resin. Most proteins in the feed stream (e.g. soybean storage proteins) have a pl higher than that of KTI and the typical pH of the feed. Therefore, these proteins typically bind more tightly to the resin. A KTI protein-containing fraction may be readily eluted from the ion exchange column by contacting the resin with a suitable eluant.

Alternatively, the pH of the feed may be controlled to be below the pI of KTI protein to provide retention of KTI proteins by the ion exchange resin. Other proteins (e.g. BBI proteins) are also bound to the resin. However, recovery of desired fractions may proceed by contacting the ion exchange resin with a suitable eluant for differential elution of protein fractions.

Suitable cation exchange resins include a variety of resins well-known in the art. In at least one embodiment, the ion exchange resin comprises a Poros 20 HS—a cross-linked poly(styrene-divinylbenzene) matrix which is surface coated with a polyhydroxylated polymer functionalized with sulfopropyl groups (e.g. propylsulfonic acid, (OH)₂S(O)—(CH₂)₃—) manufactured by Applied Biosystems.

Again with reference to FIG. 1D, after passage of feed 24 through the ion exchange column 30, an eluted KTI protein stream 33 is recovered. In order to further purify the KTI protein stream 33 and achieve purity of 95% or greater, the KTI in protein stream 33 may be isolated through the use of affinity chromatography (not depicted in FIG. 1 D). Affinity chromatography is a purification technique that typically offers purities >95% in a single step. It utilizes a specific native or added property of the target molecule to isolate it from all contaminants in the sample. In order to purify the KTI protein stream 33, the remaining contaminants that have not been removed in earlier steps, mainly soy lectin, can easily be removed though affinity chromatography. Any ready-made application specific resin (i.e. a resin having ligands attached to its surface which are specific for the compounds to be separated) can be used and equilibrated, for example, a column of N-acetyl-D-galactosamine resin (cat#A2787, Sigma Chemical Co., St. Louis, Mo.). The KTI protein stream 33 is adjusted to the same buffer composition of the resin using methods known in the art, and applied to the affinity column, whereby the contaminants adhere to the resin, and the flow-thru is collected as the purified KTI fraction.

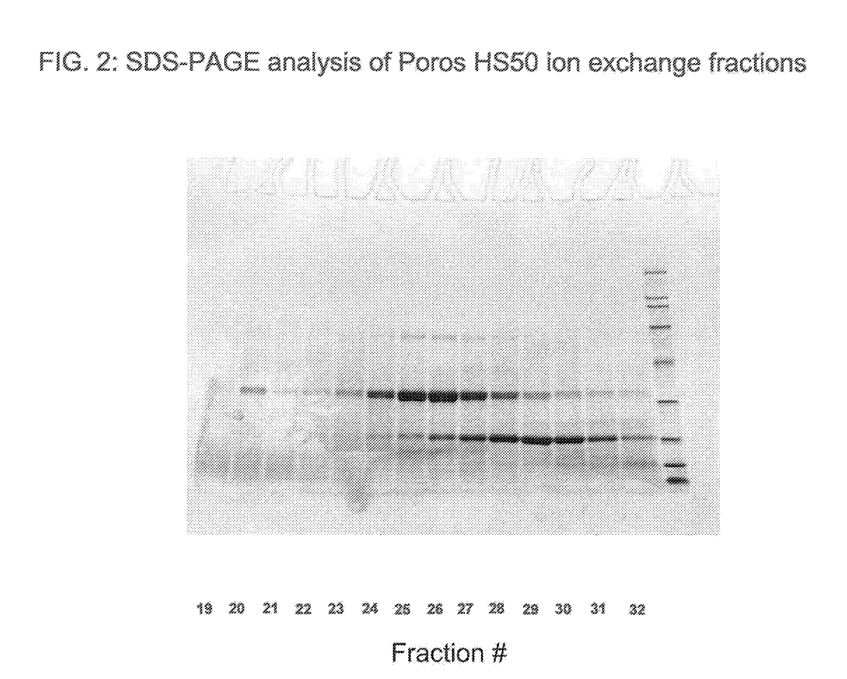

FIG. 2 illustrates the SDS-PAGE purity analysis of the various retentates and permeates isolated during the process of the invention as depicted in FIG. 1D, including the KTI protein stream 33. Lane 1 depicts the composition of the soy whey prior to separation and indicates the presence of multiple components. In contrast, Lane 5 depicts the enriched KTI fraction (i.e. the KTI protein stream 33) following the multiple separation operations illustrated in FIG. 1D. The presence of two bands indicates that KTI protein and one other contaminant (which is presumed to be soy lectin based on molecular weight of the KTI fraction) have been isolated from the soy whey following the separation processes of FIG. 1D. Removal of the remaining contaminants (i.e. soy lectin) can be accomplished as set forth in the Examples though affinity chromatography in order to obtain a KTI product that is virtually free of additional components and which has a high level of purity (>95%).

The process schemes depicted in FIGS. 1A, 1B, 1C, and 1D are not limited to the starting material used or to the order of separation and recovery of components of the soy whey set forth above, and may be utilized to prepare process streams differing from those discussed above including, for example, as set forth in the appended claims.

### E. Methods of Making KTI

In certain embodiments, a KTI protein of the invention is produced by, for example, recombinant means or synthetically. Recombinant production of a protein of the invention is done using standard techniques known by one of ordinary skill in the art. Such methods include, for example, producing a one or more coding nucleic acid sequences, which can be done by polymerase chain reaction (PCR) based methods using as a template the full-length cDNA sequence. Following production of the desired nucleic acid sequence, the sequence is inserted into an expression plasmid (including, for example, Escherichia coli pCAL-n expression plasmid), which is then transfected in a microorganism; then selection of clones containing a plasmid containing the desired sequence using selection markers (including, for example, an antibiotic resistance selection marker or a luminescent selection marker) is performed; followed by mass producing clones containing a plasmid containing the desired sequence; and purifying peptides from the desired clones (see, for example, methods described Gorlatov et al. Biochemistry (2002) 41, 4107-4116; U.S. Pat. No, 4,980,456). Alternatively peptides of the invention can be made by synthetic means or semi-synthetic means (e.g., a combination of recombinant production and synthetic means).

Synthetic production can be done by, for example, applying a fluorenylmethyloxycarbonyl(FMOC)-protective group strategy according to Carpino L. A. and Han. G Y, J. (Amer. Chem. Soc. 1981; 37; 3404-3409) or a tert-butoxycarbonyl(t-Boc)-protective group strategy. Peptides are synthesized, for example, by means of a solid-phase peptide synthesis according to Merrifield R. B. (J. Amer. Chem. Soc. 1963; 85, 2149-2154), using a multiple peptide synthesizer. Crude peptides are then purified.

An exemplary method for the synthetic production of a protein of the invention is described in the following passage. 100 mg Tentagel-S-RAM (Rapp-Polymere) at a load of 0.24 mmol/g is transferred to a commercially available peptide synthesis device (PSMM(Shimadzu)), wherein the peptide sequence is constructed step-by-step according to the carbodiimide/HOBt method. The FMOC-amino acid derivatives are pre-activated by adding a 5-fold equimolar excess of di-isopropy-carbodiimide (DIC), di-isopropy-ethylamine (DIPEA) and hydroxybenzotriazole (HOBt), and following their transfer into the reaction vessel, mixed with the resin support for 30 minutes. Washing steps are carried out by, for example, additions of DMF and thorough mixing for 1 minute. Cleavage steps are carried out by, for example, the addition of piperidine in DMF and thorough mixing for 4 minutes. Removal of the individual reaction and wash solutions is effected by forcing the solutions through the bottom frit of the reaction vessel. The amino acid derivatives FMOC-Ala, FMOC-Arg(Pbf), FMOC-Asp, FMOC-Gly, FMOC-His(Trt), FMOC-IIe, FMOC-Leu, FMOC-Lys(BOC), FMOC-Pro, FMOC-Ser(tBu) and FMOC-Tyr(tBu) (Orpegen) are employed. When synthesis is completed the peptide resin is dried. The peptide amide is subsequently cleaved off by treatment with trifluoracetic acid/TIS/EDT/water (95:2:2:1 vol) for 2 hours at room temperature. By way of filtration, concentration of the solution and precipitation by the addition of ice-cold diethyl ether, the crude product is obtained as a solid. The peptide is then purified by RP-HPLC in 0.1% TFA with a gradient of 5 on 60% acetonitrile in 40 minutes at a flow rate of 12 ml/min and evaluation of the elutant by means of a UV detector at 215 nm. The purity of the individual fractions is determined by analytical RP-HPLC and mass spectrometry.

### F. Methods of Use

In various aspects of the invention, a KTI protein of the present invention can be used to promote skin health. Examples of promoting skin health include reduction in skin discoloration or pigmentation, reduction in inflammation, wrinkle minimization, wrinkle removal, decoloring, coloring, skin softening, skin smoothing, depilation, and cleansing. Other examples of skin health and experimental methods associated therewith can be found in U.S. Patent Application Publication Nos. 20100093028, 20100092409, 20090111160, 20080249029, U.S. Pat. No. 6,555,143 (see also Chen et al., Photochemistry and Photobiology, 2008, 84:1551-1559 and Paine et al. J. Invest. Dermatol. 2001 April; 116(4):587-95).

### DEFINITIONS

To facilitate understanding of the invention, several terms are defined below.

The term “acid soluble” as used herein refers to a substance having a solubility of at least about 80% with a concentration of 10 grams per liter (g/L) in an aqueous medium having a pH of from about 2 to about 7.

The terms “soy protein isolate” or “isolated soy protein,” as used herein, refer to a soy material having a protein content of at least about 90% soy protein on a moisture free basis.

The term “subject” or “subjects” as used herein refers to a mammal (preferably a human), bird, fish, reptile, or amphibian, in need of treatment for a pathological state, which pathological state includes, but is not limited to, diseases associated with muscle, uncontrolled cell growth, autoimmune diseases, and cancer.

The term “processing stream” as used herein refers to the secondary or incidental product derived from the process of refining a whole legume or oilseed, including an aqueous or solvent stream, which includes, for example, an aqueous soy extract stream, an aqueous soymilk extract stream, an aqueous soy whey stream, an aqueous soy molasses stream, an aqueous soy protein concentrate soy molasses stream, an aqueous soy permeate stream, and an aqueous tofu whey stream, and additionally includes soy whey protein, for example, in both liquid and dry powder form, that can be recovered as an intermediate product in accordance with the methods disclosed herein.

The term “soy whey protein” as used herein refers to protein soluble at those pHs where soy storage proteins are typically insoluble, and includes but is not limited to BBI, KTI, lunasin, lipoxygenase, dehydrins, lectins, peptides, and combinations thereof, and further encompasses storage proteins.

Other proteins referred to throughout the application are defined as including but not limited to: lunasin, lectins, dehydrins, lipoxygenase, and combinations thereof.

Soy oligosaccharides are defined as including but not limited to sugar. Sugar is defined as including but not limited to sucrose, raffinose, stachyose, verbascose, monosaccharides, and combinations thereof.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above compounds, products and methods without departing from the scope of the invention, it is intended that all matter contained in the above description and in the examples given below, shall be interpreted as illustrative and not in a limiting sense.

### EXAMPLES

### Example 1

### Recovery of KTI Protein from Soy Whey Protein

Aqueous soy whey (145 l) having a total solids content of approximately 3.7 wt. % and a total protein content of 22.5 wt. % (dry wt. basis) was introduced into an OPTISEP 7000 filtration module containing a BTS-25 or MMM 0.45 micron microfiltration membrane. Passage of the aqueous soy whey through the membrane formed a permeate (132 l having a solids content of 3.2 wt. %) containing aqueous soy whey and a retentate containing greater than 99% of the initial bacteria content of the soy whey and greater than 90% of the silicon defoamer content of the soy whey.

TABLE 1
Description of Membranes Used During Experiments

| Membrane | | | | | |
|---|---|---|---|---|---|
| Name | Membrane Type | Material | Pore Size | | Manufacturer |
| BTS-25 | Microfiltration (MF) | Polysulfone (PS) | 0.5 | μm | Pall |
| MMM 0.45 μm | Microfiltration (MF) | Modified | 0.45 | μm | Pall |
| | | Polysulfone (MPS) | | | |
| RC 100 kDa | Ultrafiltration (UF) | Regenerated | 100 | kDa | Microdyn- |
| | | Cellulose (RC) | | | Nadir |
| PES 5 kDa | Ultrafiltration (UF) | Polyethersulfone | 5 | kDa | Microdyn- |
| | | (PES) | | | Nadir |

| | | | | |
|---|---|---|---|---|
| NF PES 30 | Nanofiltration (NF) | Polyethersulfone | 30% NaCl Rejection | Microdyn- |
| | | (PES) | | Nadir |
| NF 20 | Nanofiltration (NF) | Polysulfone Thin | 35% NaCl Rejection | Sepro |
| | | Film | | |
| SG | Reverse Osmosis | Thin Film | 98.2% NaCl Rejection | GE |
| | (RO) | | | |

Permeate (132 l) from the microfiltration module was introduced into an OPTISEP 7000 filtration module containing a regenerated cellulose (RC) ultrafiltration membrane having a pore size of approximately 100 kDa. Passage of the permeate through the ultrafiltration membrane formed a second permeate containing sugars, minerals, and vitamins, and a second retentate (approx. 2 l) having a solids content of approximately 25.4 wt. % and a total soy protein content of approximately 83 wt. % (dry basis).

Second retentate was dried via lyophilization using a Virtis Freezemobile 25XL. An aliquot of dried sample (53 mg) was slurried in water to a final solids concentration of 25%, then incubated with mixing at room temperature for 10 min. to facilitate solubilization. Insoluble material was removed with centrifugation at 2000×g for 10 min, and the supernatant further diluted 5× with 20 mM sodium citrate, pH 3.0. The resultant pH drop led to precipitation of some of the components of the mixture, which were subsequently removed with centrifugation at 2000×g for 5 min.

A 7×25 mm Poros 50 HS column (0.8 ml bed volume; Applied Biosystems) was pre-equilibrated in 20 mM sodium citrate, pH 3.0 buffer. Sample was filtered thru a 0.45 micron syringe filter, and 0.5 ml applied to the column at a flow rate of 1 ml/mm. Unbound protein was washed free of the column with 20 column volumes of equilibration buffer, then bound fractions recovered in 1 ml fractions using a 40 column volume, 0-100% linear gradient of 1M sodium chloride in 20 mM sodium citrate buffer, pH 3.0. Coomassie-stained SDS-PAGE gels were used to analyze each fraction. KTIₐₚₚ was localized to fractions 25-32 (see FIG. 2) based on MWₐₚₚ. Fractions 29 and 30 were pooled and assayed for total protein using the modified Lowry protein assay (Sigma-Aldrich Total Protein Kit, Micro-Lowry, Onishi and Barr Modification). KTI activity was estimated using the procedure of Kakade et al (Kakade, M. L., Simons, N., and Liener, I. E., 1969. An Evaluation of Natural vs. Synthetic Substrates for Measuring the Antitryptic Activity of Soybeans. Cereal Chem. 46:518). The protein concentration of pooled samples was found to be 0.48 mg/ml, and specific activity to be 708 Ti units/g protein. The enriched KTI sample was also assayed for chymotrypsin inhibition activity (using Schechter, N., Sprows, J., Schoenberger, O., Lazarus, G., Cooperman, B., and Rubin, H., 1989. Reaction of Human Skin Chymotrypsin-like Proteinase Chymase with Plasma Proteinase Inhibitors. J. Biol, Chem. 264: 21308-21315.Jameson, G. W., Roberts, D. V., Adams, R. W., Kyle, W. S. A., and Elmore, D. T., 1973. Determination of the Operational Molarity of Solutions of Bovine α-Chymotrypsin, Trypsin, Thrombin and Factor X_{α} by Spectrofluorimetric Titration. Biochem. J. 131: 107-117.) and found to contain essentially none. Thus, KTI prepared as described in this example was free of contaminating BBI activity.

SDS-PAGE analysis of the various fractions prepared during KTI purification is depicted in FIG. 3. KTI of >95% purity can be obtained using affinity chromatography to remove the remaining contaminant shown in lane 5. Thus, a column of N-acetyl-D-galactosamine resin (cat#A2787, Sigma Chemical Co., St. Louis, Mo.) would be equilibrated in 20 mM Tris, 154 mM NaCl, pH 7.4. The enriched KTI fraction would be adjusted to this same buffer composition using methods known in the art, and applied to the affinity column. Contaminant would bind to the resin, and the column flow-thru would be collected as the purified KTI fraction.

### Example 2

### Confirmation of KTI Identity by Mass Spectrometry

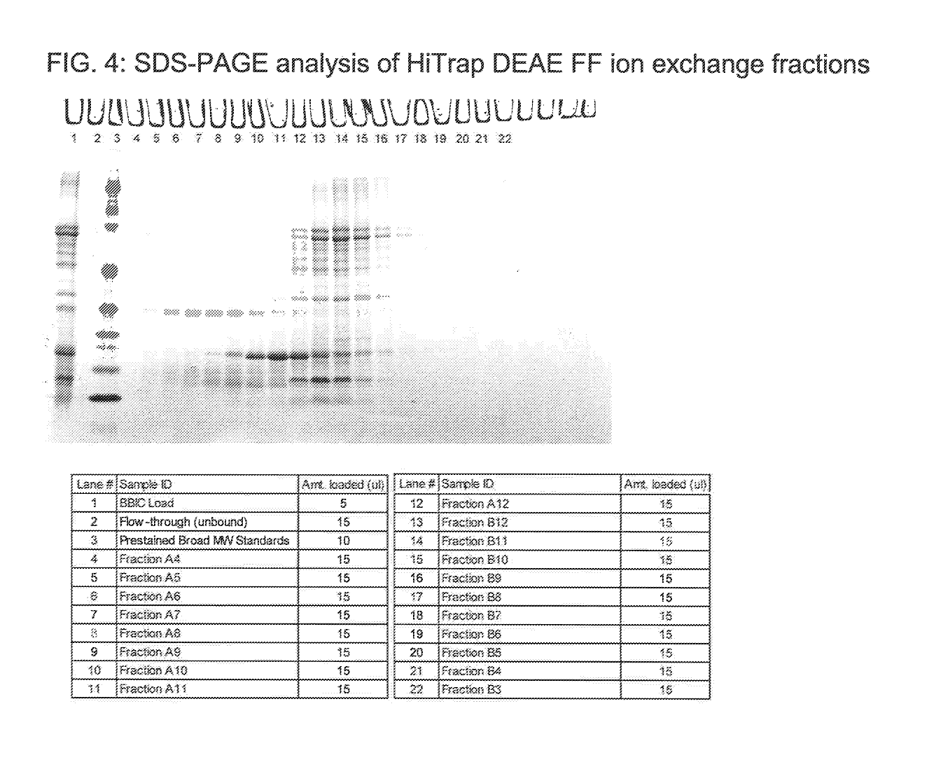

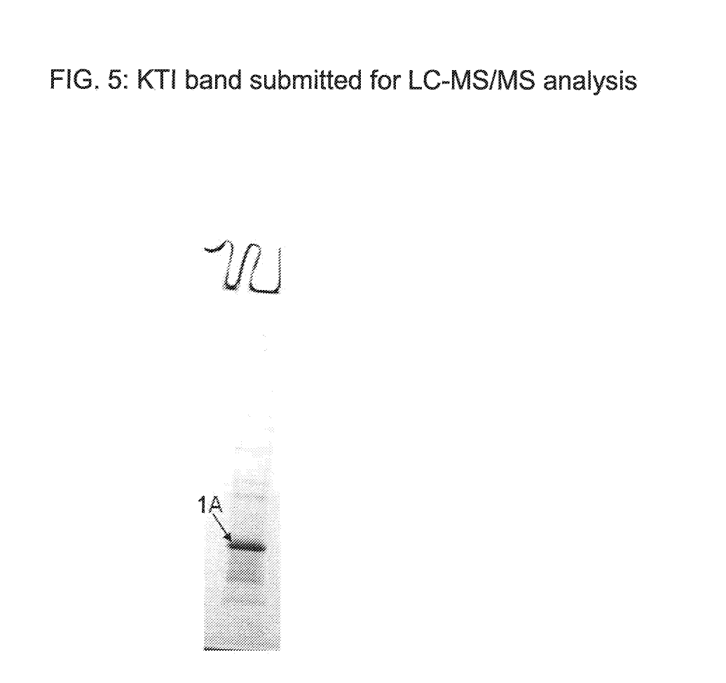

KTI was purified to approx. 90% purity (based on SDS-PAGE analysis) using the following procedure. 1 gram of soy Bowman-Birk Inhibitor Concentrate (BBIC, Central Soya Co. Lot#02176-2) was resuspended in 20 ml of 20 mM HEPES buffer at pH 6.8 to yield a 5% (w/v) slurry. The slurry was stirred in a beaker for 20 min at room temperature on a stir plate using a magnetic stirring rod to completely hydrate the material, then centrifuged at 10.000×g for 10 min to remove insoluble material. The resultant supernatant was decanted and filtered thru a 0.2 μm filter, and 1 ml of the filtrate diluted to 10 ml with starting buffer (25 mM Tris, pH 7.5). The diluted sample was then applied directly to a 1 ml HiTrap DEAE FF column (GE Healthcare cat#17-5055-01) equilibrated in starting buffer at a flow rate of 1 ml/min. After washing unbound proteins free, the column was eluted with a linear, 40 column volume gradient of 0-1 M NaCl in 20 mM Tris, pH 7.5. One ml fractions were collected during elution, and analyzed via SDS-PAGE (see FIG. 4). KTIₐₚₚ was identified in fractions 8-15. Fraction All contained approx. 90% pure KTI, and was chosen for analysis via Mass Spectrometry. Thus, 12.5 μls of this fraction were separated on a 10-20% SDS-PAGE gel, coomassie stained, and submitted for Mass Spectrometry analysis (see FIG. 5).

Analyses were performed at the Donald Danforth Plant Sciences Center (St. Louis, Mo.). Thus, an in-gel tryptic digest procedure was used to fragment the KTIₐₚₚ band into peptides, which were then subjected to LC-MS/MS analysis using a QStar XL nanospray-QTOF instrument (Applied Biosciences), Spectra were searched against both the NCBInr and TIGR glycine max databases to identify the proteins from which the trypic peptides originated. Band 1A was identified as trypsin inhibitor subtype A from Glycine max, FIG. 5, Specific peptides identified were NELDKGIGTIISSPYR (aa 73-88), FIAEGHPLSLK (aa 91-101), IGENKDAMDGWFR (aa 132-144), VSDDEFNNYK (aa 148-157), and NKPLVVQFQK (aa 191-200) from KTI.

Example 3

### Purification of KTI from Bulk Soy Whey Protein using Expanded Bed Adsorption (EBA) Chromatography

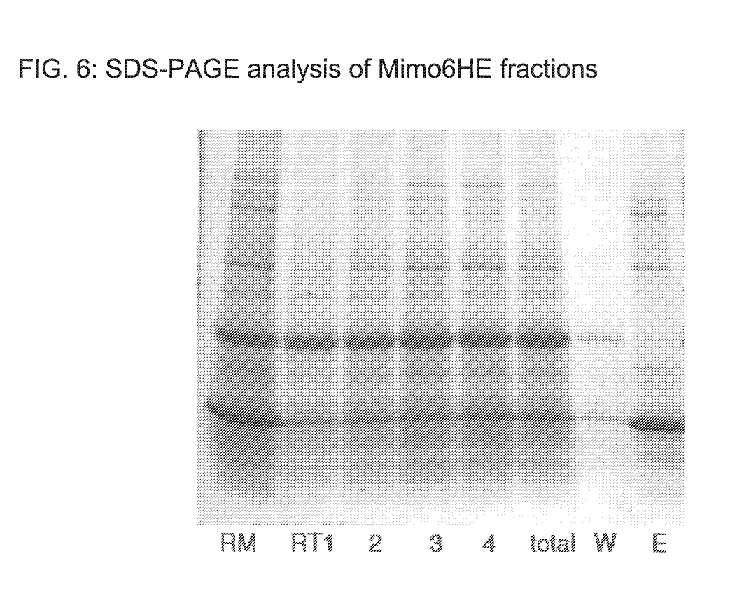

200 ml of aqueous soy whey having a total solids content of 1.92%, was adjusted to pH 6.0 with sodium hydroxide and applied to a 1×25 cm column of Mimo6HE resin (UpFront Chromatography, Copenhagen Denmark) equilibrated in 10 mM sodium citrate, pH 6.0. The material was loaded onto the column from the bottom up at 20° C.-25° C. using a linear flow rate of 7.5 cm/min. Samples of column flow-through were collected at regular intervals for later analysis. Unbound material was washed free of the column with 10 column volumes of equilibration buffer, the bound material was then recovered by elution with 30 mM sodium hydroxide. 10 μls of each fraction recovered during EBA chromatography of a suspension of soy whey powder were separated on a 4-12% polyacrylamide gel and stained with Coomassie Brilliant Blue R 250 stain. SDS-PAGE analysis of the column load, flow-through, wash, and sodium hydroxide eluate samples is depicted in FIG. 6. As used in FIG. 6, RM: raw material (column load); RT1-4: flow-through material (run through) collected at equal intervals during the load; total: the total run-through fraction; W: column wash; E: column eluate. The bulk of the loaded protein was clearly seen eluting in the flow-through, while the bulk of the KTI protein remained bound to the resin. A total of 355 mg of protein, the bulk of which is KTI, was recovered in the eluate, for a yield of 1.8 mg/ml of starting material. Under these conditions, the capacity of this resin was shown to be 17.8 mg of KTI (plus minor contaminants) per ml of adsorbant.

To achieve purity of 95% or greater, the material will be lyophilized, then resolubilized in a minimal volume of buffer (e.g., 20 mM Tris, pH 7.5 containing 100 mM NaCl) and applied to a Superose 6 Size exclusion chromatography column equilibrated in the same buffer. Column will be developed at a flow rate of 0.5 ml per minute, and the 280 nm wavelength absorbance of the eluate continuously monitored. Fraction collection will start immediately following elution of one column void volume, and the purified KTI localized to specific fractions using SDS-PAGE. KTI-containing fractions will be pooled and assayed for trypsin inhibition to verify purity.

One skilled in the art would readily appreciate that the methods and compositions described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A process for separating and purifying a KTI product having a total KTI protein concentration of at least 95 wt. %, wherein the process comprises passing a soy protein processing stream through at least one separation operation to isolate a purified KTI protein stream.

2. The process of claim 1, wherein the at least one separation operation is selected from the group consisting of: membrane separation, chromatographic separation, electrophoresis, dialysis, particulate filtration, precipitation, centrifugation, crystallization, gravity separation, and any combination thereof.

3. The process of claim 1, wherein the at least one separation operation is membrane separation.

4. The process of claim 3, wherein the soy processing stream is passed through at least one separation membrane at a volumetric flow of at least about 1 liters fluid/hour-m².

5. The process of claim 4 wherein the volumetric flow is from about 1 to about 150 liters fluid/hour-m².

6. The process of claim 4, wherein the soy processing stream is passed through the at least one separation membrane at a temperature of from about 0° C. to about 100° C.

7. The process of claim 4, wherein the soy processing stream is passed through the at least one separation membrane at a temperature of from about 25° C. to about 75° C.

8. The process of claim 4, wherein the at least one separation membrane comprises a microfiltration membrane for removing impurities from the soy processing stream.

9. The process of claim 8, wherein the pore size of the microfiltration membrane is between about 0.1 μm and about 20 μm.

10. The process of claim 3, wherein the at least one separation operation further comprises chromatographic separation.

11. The process of claim 10, wherein the chromatographic separation is ion exchange chromatography and affinity chromatography.

12. The process of claim 1, wherein the purified KTI product comprises an amino acid sequence at least 80% identical to SEQ ID NO: 1.

13. The process of claim 1, wherein the purified KTI product comprises an amino acid sequence at least 85% identical to SEQ ID NO: 1.

14. The process of claim 1, wherein the purified KTI product comprises an amino acid sequence at least 90% identical to SEQ ID NO: 1.

15. The process of claim 1, wherein the purified KTI product comprises an amino acid sequence at least 95% identical to SEQ ID NO: 1.

16. A process for separating and purifying a KTI product having a total KTI protein concentration of at least 95 wt. %, wherein the process comprises:
(a) passing a soy processing stream containing soy proteins and impurities, wherein the impurities are comprised of soy storage proteins, microorganisms, minerals, and sugars, through at least one separation technique to form a first permeate and a first retentate, the first permeate comprising the soy proteins, and the retentate comprising the impurities;
(b) passing the first permeate through at least one separation operation to form a second permeate and a second retentate, the second retentate comprising a significant fraction of proteins and the second permeate comprising impurities;
(c) combining the second retentate with a carrier stream for passage through at least one separation operation to isolate a KTI protein stream from other proteins in the processing stream to form a partially purified KTI product; and
(d) passing the partially purified KTI product through at least one chromatographic separation operation to form a purified KTI product having a total KTI protein concentration of at least 95 wt %.
